(19) United States
(12) Patent Application Publication
(10) Pub. No.: US 2026/0306225 A1
(43) Pub. Date: Oct. 8, 2026

(54) METHOD OF CONTROLLING THE BIODEGRADABILITY OF MAGNESIUM AND ITS ALLOYS

(71) Applicant: OsseoLabs Co., Ltd., Bangkok (TH)

(72) Inventors: Patcharapit Promoppatum, Nonthaburi (TH); Suwitchaya Amapites, Rayong (TH); Vikram Ahuja, Bangkok (TH); Ola Harrysson, Raleigh, NC (US)

(21) Appl. No.: 19/640,736

(22) Filed: Apr. 7, 2026

Related U.S. Application Data
(60) Provisional application No. 63/784,381, filed on Apr. 7, 2025.

(51) Int. Cl. A61C 13/00; A61C 8/00; A61K 6/84; G06F 30/23; G06F 111/10; G06F 119/18

(52) U.S. Cl. CPC A61C 13/0004; A61C 8/0015; A61K 6/84; G06F 30/23; G06F 2111/10; G06F 2119/18

(57) ABSTRACT

A method for manufacturing a biodegradable magnesium alloy implant with a controlled degradation rate includes defining a bone remodeling time target based on the implant's anatomical location and physiological requirements. A computational numerical model applying a convection-diffusion equation and Fick's first law calculates an optimal oxide layer thickness to achieve a predicted degradation time equal to or greater than the time target. The numerical model integrates the grain size of the magnesium alloy to counteract micro-galvanic corrosion. The implant surface is modified by a high-temperature oxidation process at a temperature between 400° C. and 600° C. to form the calculated optimal oxide layer thickness. Subsequently, the surface-modified implant is cooled at a controlled rate of 1° C. to 15° C. per minute to prevent micro-cracking within the oxide layer. The method provides tailored implants, such as dental meshes and fusion prostheses, that maintain mechanical integrity throughout the healing period.

### CROSS-REFERENCE TO RELATED APPLICATION

The present application claims the benefit of US provisional patent application No. 63/784,381. The disclosures of the above-mentioned application are incorporated herein by reference in their entirety.

### FIELD OF THE INVENTION

The present invention relates generally to the field of biomedical engineering, materials science, and medical implants. More specifically, it relates to an advanced, predictive methodology for manufacturing biodegradable magnesium alloy implants with a precisely controlled degradation rate by integrating computational numerical modeling with a strictly controlled high-temperature oxidation (HTO) process and defined cooling parameters.

### BACKGROUND OF THE INVENTION

The foundation of modern orthopedic and maxillofacial reconstructive surgery relies heavily on metallic implants to replace, support, or augment biological structures. Historically, bio-inert permanent metals such as titanium alloys, stainless steel, and cobalt-chromium alloys have been the standard of care due to their exceptional mechanical strength and biocompatibility. However, permanent implants inherently present significant long-term clinical challenges. The mismatch in the elastic modulus between rigid permanent metals and human cortical bone leads to a detrimental phenomenon known as “stress shielding,” wherein the implant bears the majority of the physiological load, causing the surrounding natural bone to weaken and resorb over time. Furthermore, in applications requiring only temporary fixation—such as fracture plates, screws, or dental meshes—the permanent nature of these implants frequently necessitates a secondary surgical procedure for removal once the tissue has healed. This additional intervention increases patient morbidity, introduces further risks of surgical complications and infections, and imposes a substantial economic burden on healthcare systems.

To resolve these limitations, biodegradable metals, particularly magnesium (Mg) and its alloys, have emerged as revolutionary biomaterials. Magnesium possesses an elastic modulus and density that closely approximate those of natural bone, thereby mitigating stress shielding. More importantly, magnesium degrades in vivo into non-toxic products that can be naturally metabolized and excreted, effectively eliminating the need for secondary removal surgeries. Furthermore, the release of magnesium ions has been shown to actively stimulate osteogenesis, enhancing the bone healing process. Despite their extraordinary potential, the clinical translation of magnesium-based implants is heavily hindered by a critical flaw: their inherently rapid and uncontrolled degradation rate in chloride-rich physiological environments. In the human body, magnesium undergoes rapid electrochemical corrosion, leading to a premature loss of the implant's mechanical integrity long before the targeted tissue or bone has sufficiently healed. This rapid biocorrosion is accompanied by localized pitting, local alkalization, and the rapid evolution of hydrogen gas pockets, which can disrupt cell proliferation, cause tissue necrosis, and ultimately lead to implant failure.

To date, attempts to control this degradation have relied predominantly on costly, time-consuming, and resource-intensive in vitro and in vivo trial-and-error experimentation. There has been a profound lack of comprehensive predictive capabilities that can reliably tailor the degradation kinetics of an implant to match the exact healing time target required for specific, varying clinical applications. The origin and core conceptual framework of the present invention emerge directly from the critical need to shift from this unpredictable trial-and-error paradigm to an in silico predictive design methodology.

The present invention introduces a groundbreaking method that actively dictates the parameters of a surface modification process based on computational prediction. The concept relies on defining a specific patient or anatomical “bone remodeling time target (T_{b})”. A computational numerical model—utilizing convection-diffusion equations and Fick's first law—is then employed to inversely calculate the precise optimal oxide layer thickness (tₒₓ) required to achieve a degradation time that meets or exceeds T_{b}. Crucially, this model uniquely integrates the grain size of the magnesium alloy as a controlling parameter to counteract micro-galvanic corrosion. The physical implant is then subjected to a highly specific High-Temperature Oxidation (HTO) process at 400° C. to 600° C. to grow exactly the calculated oxide thickness, followed by a precisely controlled cooling rate of 1° C. to 15° C. per minute to prevent micro-cracking within the newly formed oxide layer. This synergistic integration of computational prediction and controlled thermal processing ensures sustained mechanical integrity tailored to specific clinical time targets.

In the ongoing effort to optimize biodegradable magnesium alloys for medical applications, numerous strategies have been explored in the prior art. These strategies generally fall into categories such as bulk compositional modification (alloying), unconventional physical fabrication, mechanical topology optimization, or empirical wet-chemical surface coatings. However, they consistently fail to provide the predictive, dynamically tailored degradation control offered by the present invention.

A significant portion of the prior art focuses on altering the bulk chemical composition of the magnesium alloy to slow down corrosion, specifically through the addition or removal of rare-earth elements (REEs). For instance, European Patent Application EP2169090A1 and its corresponding US Patent Application US20170157300A1 disclose a biodegradable magnesium alloy utilizing specific mixtures of heavy rare-earth elements (HREs) such as Ho, Er, Lu, Tb, and Tm. The primary objective of these documents is to reduce the formation of hard intermetallic precipitates to improve the thermo-mechanical processability of the alloy, making it easier to manufacture drawn tubes for stents. Conversely, European Patent Application EP4272774A1 takes the opposite approach by disclosing a biodegradable magnesium alloy entirely free of rare-earth elements, relying instead on a precise composition of Zn, Mn, Ca, Sr, Sn, and Zr, coupled with extensive mechanical processing (hot extrusion and rolling) to physically refine the grain size. While these compositional and metallurgical approaches improve generalized corrosion resistance and structural strength, they provide only static, “one-component-fits-all” material solutions. They do not offer any mechanism to predict or adjust the degradation timeline to match specific, varying patient clinical needs (e.g., T_{b}). In stark contrast, the present invention is not limited to a single static alloy composition but provides a universal predictive methodology. Instead of relying solely on the internal metallurgy, the present invention utilizes a computational model that dynamically calculates the optimal external oxide layer thickness customized entirely around a pre-determined biological time target.

Another area of prior art involves unconventional fabrication states, such as supersaturation. U.S. Pat. No. 8,435,281B2 describes a bioerodible medical device incorporating a supersaturated magnesium alloy created through rapid solidification processes to minimize microgalvanic couples. Critically, U.S. Pat. No. 8,435,281B2 strictly mandates that any subsequent processing must avoid high temperatures to prevent the material from reverting to its equilibrium condition, explicitly stating that processing must occur at temperatures lower than 140° C. This teaches directly away from the present invention. The present invention specifically requires subjecting the magnesium alloy to a High-Temperature Oxidation (HTO) process at target temperatures between 400° C. and 600° C. to deliberately form a protective, calculated oxide layer (tₒₓ). Thus, the present invention operates on fundamentally different thermodynamic and manufacturing principles.

Furthermore, advancements in 3D printing and structural design have been explored to address mechanical mismatches. U.S. Pat. No. 11,396,687B2 discloses specialized metal-containing feedstocks for additive manufacturing that are intentionally enriched with high-vapor-pressure metals (like Mg and Zn) to compensate for the vaporization that occurs in the melt pool during laser printing. Similarly, United States Patent Application US20170014169A1 discloses a method for designing patient-specific implants using topology optimization to adjust external and internal porous geometries. By 3D printing porous structures (specifically focusing on Nitinol), the physical stiffness of the implant is reduced to match the patient's cortical bone, thereby preventing stress shielding. While both US11396687B2 and US20170014169A1 represent significant advancements in mechanical design and the fabrication of complex structures, they completely fail to address the electrochemical degradation timeline of magnesium in vivo. Even a perfectly stiffness-matched, flawlessly 3D-printed magnesium implant will still be subject to rapid, uncontrolled biocorrosion. The present invention operates beyond initial physical fabrication. It provides the critical missing step: employing predictive convection-diffusion modeling and precise thermal oxidation to protect the fabricated geometry from premature degradation.

Finally, regarding surface treatments, European Patent Application EP2204196A1 discloses a method for forming a corrosion-resistant film (such as calcium phosphate) on a magnesium base material by immersing it in a treatment solution. The core feature of EP2204196A1 is controlling the flow speed of the solution relative to the base material to ensure the formed film has a minimal variation in surface hardness (dispersion value of Vickers hardness less than 21). This represents a purely empirical, wet-chemical approach based on fluid dynamics. It lacks any computational modeling capability to predict the required film thickness for a specific bone remodeling target. In contrast, the present invention does not rely on wet-chemical solution flow. Instead, it utilizes a thermal gas-phase HTO process governed by predictive mathematical modeling. Furthermore, the present invention uniquely identifies and solves the problem of thermal shock and micro-cracking inherent in thermal oxide layers by implementing a precisely calculated cooling rate of 1° C. to 15° C. per minute—a critical engineering parameter entirely absent from EP2204196A1 or any wet-chemical methodology.

In conclusion, while the prior art demonstrates noble efforts to improve magnesium alloys through rigid compositional restrictions, low-temperature supersaturation, mechanical topology optimization, or empirical wet-chemical coatings, none provide a comprehensive framework to accurately predict and actively control the degradation timeline in vivo. There remains a critical, unmet need for a methodology that eliminates trial-and-error experimentation and actively synchronizes the lifespan of the implant with the biological tissue remodeling process. The present invention bridges this gap by offering a transformative, computational modeling-driven HTO method, incorporating precise grain size analysis and controlled cooling mechanisms, to ensure sustained mechanical integrity tailored to specific clinical time targets.

### SUMMARY OF THE INVENTION

The present invention provides a comprehensive, predictive methodology, a system, and a resulting surface-modified medical implant that effectively overcome the limitations of rapid and uncontrolled biodegradation inherent in magnesium and its alloys. A primary object of the present disclosure is to eliminate the reliance on resource-intensive in vitro and in vivo trial-and-error experimentation by providing an in silico predictive design framework that synchronizes the implant's degradation profile precisely with a patient-specific tissue healing timeline.

In one aspect, the present disclosure provides a method of manufacturing a biodegradable magnesium alloy implant with a controlled degradation rate. The method comprises defining a bone remodeling time target (T_{b}) based on an anatomical location and a physiological load-bearing requirement of the implant. The surface of the magnesium alloy implant is modified by subjecting the implant to a high-temperature oxidation (HTO) process at a target temperature between 400° C. and 600° C. to form a protective oxide layer. Characteristically, the method further comprises calculating an optimal oxide layer thickness (tₒₓ) required to achieve a predicted implant degradation time (T_{d}) that is equal to or greater than the time target (T_{b}). This calculation utilizes a computational numerical model applying a convection-diffusion equation and Fick's first law to simulate a mass transfer of magnesium ions. Uniquely, the numerical model integrates the grain size of the magnesium alloy as a controlling parameter to counteract micro-galvanic corrosion. Following the calculation, the HTO process is executed to generate the oxide layer having exactly the calculated optimal oxide layer thickness (tₒₓ). Subsequently, the surface-modified implant is cooled from the target temperature at a precisely controlled cooling rate of 1° C. to 15° C. per minute to prevent micro-cracking within the formed oxide layer.

In certain embodiments of the method, the simulated mass transfer in the computational numerical model incorporates dynamic concentration tracking the degradation front, an initial solid concentration of magnesium (c_{solid}), and a saturation concentration of magnesium ions (cₛₐₜ) in a simulated body fluid at a physiological temperature of 37° C. and a pH of 7.4. Calculating the optimal oxide layer thickness (tₒₓ) may comprise an inverse design algorithm iteratively adjusting an oxide layer thickness parameter within the computational numerical model until the predicted implant degradation time (T_{d}) aligns with the bone remodeling time target (T_{b}). Furthermore, the computational numerical model may continuously correlate the grain size with a micro-galvanic corrosion rate factor to dynamically adjust the required optimal oxide layer thickness. In some embodiments, the model can further generate a spatial degradation profile and a localized stress-strain distribution map of the implant.

To precisely regulate the oxidation kinetics and prevent substrate ignition, the high-temperature oxidation (HTO) process is preferably performed in a controlled gas atmosphere having a specifically restricted oxygen partial pressure combined with an argon inert gas.

The method is highly applicable to porous structures having an interconnected pore network, such as a dental mesh or a fusion prosthesis. In embodiments where the porous structure is a dental mesh, the calculated optimal oxide layer thickness (tₒₓ) restricts a hydrogen gas evolution rate to a physiologically acceptable level throughout the bone remodeling time target (T_{b}). In embodiments where the porous structure is a fusion prosthesis, the oxide layer dictates a degradation profile configured to provide a gradual transfer of physiological load to the newly formed bone, thereby actively preventing stress shielding while maintaining sufficient structural integrity.

In another aspect, the present disclosure provides a surface-modified biodegradable magnesium alloy implant comprising a magnesium alloy substrate and a protective oxide layer formed on a surface of the substrate by a high-temperature oxidation (HTO) process. The implant is manufactured by the aforementioned predictive method, wherein the protective oxide layer has a specific optimal thickness (tₒₓ) predetermined by the computational numerical model based on the grain size of the substrate. This synchronizes the degradation profile of the implant with a patient-specific bone remodeling time target (T_{b}). Furthermore, the protective oxide layer controls uniformity by the controlled cooling rate between 1° C. to 15° C. per minute. The magnesium alloy substrate may comprise a microstructure substantially free of rare-earth element (REE) inclusions to enhance biocompatibility. In a specific embodiment, the implant is a high-porosity dental mesh comprising a tailored oxide layer configured to stimulate osteogenesis while effectively suppressing rapid corrosion burst.

In yet another aspect, the present disclosure relates to computation of a biodegradable magnesium alloy implant with a customized degradation rate. A numerical model applies a convection-diffusion equation to inversely calculate an optimal oxide layer thickness (tₒₓ) utilizing the grain size of the implant as an input parameter to achieve a predefined bone remodeling time target (T_{b}). The heat treatment apparatus comprises a controller programmed to receive the calculated optimal oxide layer thickness (tₒₓ), regulate the HTO process duration to form said thickness, and engage a cooling mechanism to strictly cool the implant at a rate between 1° C. to 15° C. per minute. To facilitate personalized medicine, the biodegradable device may be further configured to support patient-specific medical devices from imaging data, such as CT or MRI. Patient-specific medical devices can be designed based on patient's bone anatomy and pre-define bone remodeling time target (T_{b}) that can support sufficient bone healing.

### BRIEF DESCRIPTION OF THE DRAWINGS

FIG. 1 illustrates the Method of controlling biodegradability of magnesium and its alloys.

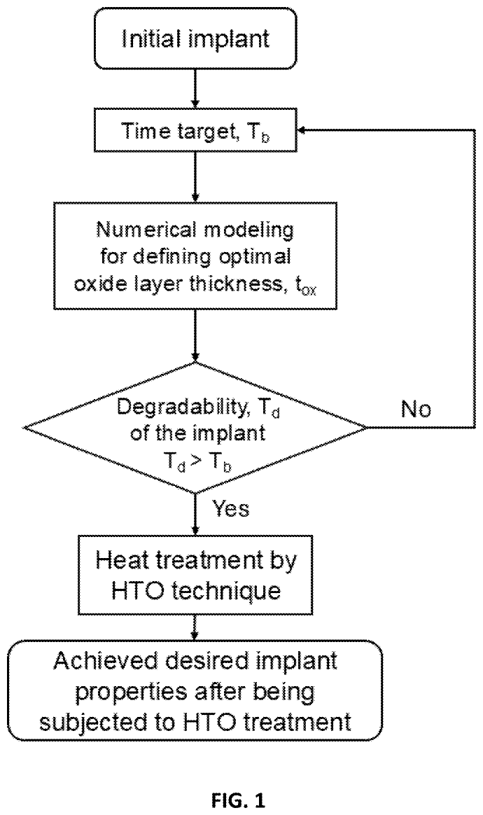

FIG. 2 illustrates the controlling biodegradability of magnesium and its alloys method a). Initial implant b). Implant with microbiological environment in simulation c). Optimal heat treatment (HTO) conditions with hit time targets for bone remodeling d). Final implant model with HTO product.

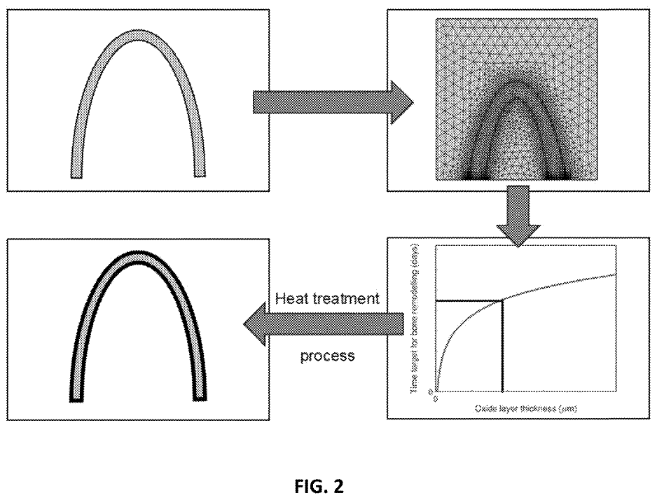

FIG. 3 illustrates the demonstration of corrosion behavior from simulation and experiment of simplified model a). Mass loss rate of the simplified model across varying oxide layer thicknesses. b). Mass loss rate of the experimental simplified specimen featuring the optimized oxide layer. c). Corrosion rate between simulating and experimental optimal simplified model. d). Observed degradation behavior of the experimental specimen.

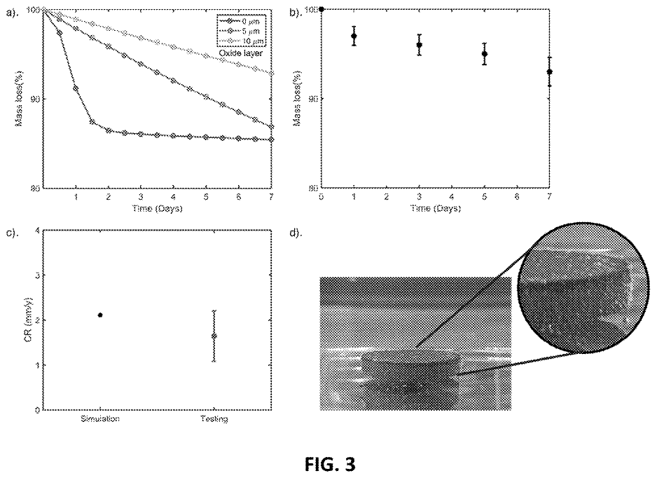

FIG. 4 illustrates of example case following the invention implementation Mesh a). Initial Model of Mg Dental Mesh b). Simplified Dental Mesh Model with Microbiological Environment in Simulation c). Simplified Dental Mesh Model with Result of Simulation Method d). Performed Mg Dental Mesh after HTO Treatment.

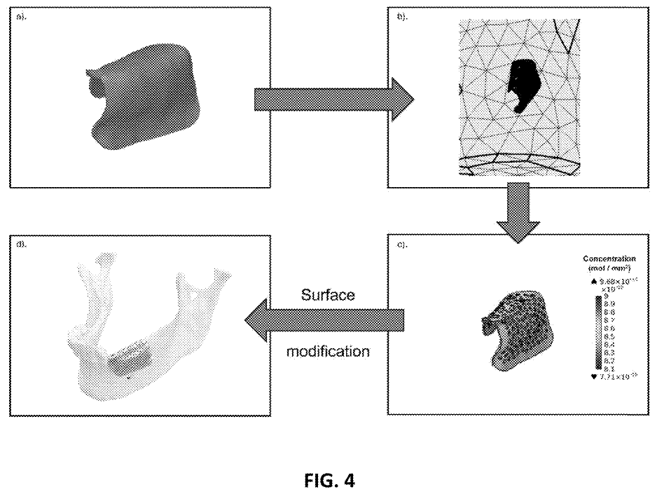

### DETAILED DESCRIPTION OF THE INVENTION

The present disclosure relates to a predictive methodology, system, and resulting surface-modified medical implants that effectively overcome the limitations of rapid and uncontrolled biodegradation inherent in magnesium (Mg) and its alloys. The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention.

### I. Definitions

As used herein, the term “biodegradable magnesium alloy” refers to any metallic alloy wherein the primary constituent is magnesium (Mg), which undergoes gradual in vivo degradation, corrosion, or resorption when exposed to physiological fluids. As used herein, the term “bone remodeling time target (T_{b})” refers to the specific patient-derived or anatomically defined duration required for the host bone to adequately heal, fuse, or remodel sufficiently to bear physiological loads without the complete structural support of the implant. As used herein, the term “predicted implant degradation time (T_{d})” refers to the computationally calculated duration during which the implant maintains its critical mechanical integrity before structural failure due to biocorrosion. As used herein, the term “optimal oxide layer thickness (tₒₓ)” refers to the exact physical thickness of the protective ceramic-like oxide layer grown on the surface of the implant, which is mathematically determined to delay degradation such that T_{d}≥T_{b}.

### II. System Architecture

The present invention encompasses a comprehensive system for manufacturing a biodegradable magnesium alloy implant with a customized degradation rate. The system essentially comprises a computing device and a specialized heat treatment apparatus communicatively coupled together. The computing device includes at least one processor, a memory unit, and customized software instructions configured to execute a computational numerical model. In a preferred embodiment, the computing device is configured to receive and convert patient-specific medical imaging data, such as Computed Tomography (CT) or Magnetic Resonance Imaging (MRI) scans, directly into the bone model, where one can predefine bone remodeling time target (T_{b}) for satisfactory bone healing. For instance, the imaging data may reveal the exact dimensions of a segmental defect, allowing the system to computationally ascertain the physiological requirement such as biological environment and load bearing, as well as the corresponding biological healing timeframe.

The heat treatment apparatus is specifically designed to execute a High-Temperature Oxidation (HTO) process. The apparatus comprises a programmable controller that receives the mathematically determined optimal oxide layer thickness (tₒₓ) from the computing device. Based on this input, the controller actively regulates the internal environmental parameters of the furnace, including the target temperature (between 400° C. and 600° C.), the duration of the thermal exposure, the oxygen partial pressure, and the flow of inert gases (such as argon). Crucially, the heat treatment apparatus also includes a precision cooling mechanism governed by the controller to strictly cool the implant at a highly specific rate of 1° C. to 15° C. per minute upon completion of the oxidation phase.

### III. Methodology for Predictive Degradation Control

The manufacturing method of the present invention represents a paradigm shift from traditional trial-and-error material testing to an in silico predictive design framework. The method operates through a series of integrated computational and physical steps:

### Step 1: Clinical Assessment and Target Definition

The process initializes by defining the bone remodeling time target (T_{b}) based on the specific anatomical location and the physiological load-bearing requirement of the magnesium alloy implant. For example, a mandibular fracture plate may require a T_{b} of 24 to 48 weeks to ensure sufficient bone union, whereas a highly porous dental mesh utilized in Guided Bone Regeneration (GBR) may require a T_{b} of 16 weeks tailored to prevent premature collapse of the soft tissue envelope.

### Step 2: Computational Numerical Modeling and Inverse Design

Once T_{b} is established, the method employs an advanced computational numerical model to calculate the optimal oxide layer thickness (tₒₓ) required to achieve a predicted implant degradation time (T_{d}) that is equal to or marginally greater than the time target (T_{b}). The numerical model, typically executed via finite element analysis (FEA) software, rigorously applies the convection-diffusion equation and Fick's first law to simulate the mass transfer of magnesium ions in a simulated physiological environment.

To ensure extreme predictive fidelity, the model incorporates a dynamic concentration tracking condition that actively tracks the retreating degradation front as the magnesium substrate dissolves over time. The governing physiological parameters embedded within the simulation include the initial solid concentration of magnesium (c_{solid}), the precise saturation concentration of magnesium ions (cₛₐₜ) within a simulated body fluid (SBF) at a standard physiological temperature of 37° C. and a buffered pH of 7.4, and the effective diffusion coefficients (D_{Mg}).

Uniquely, the present numerical model directly integrates the grain size of the magnesium alloy as a primary controlling parameter. In polycrystalline magnesium alloys, grain boundaries often act as preferential sites for micro-galvanic corrosion due to the precipitation of secondary phases or the accumulation of impurities. The computational model continuously correlates the measured grain size of the raw substrate with a micro-galvanic corrosion rate factor. By doing so, the algorithm dynamically adjusts the predicted mass loss rate. Smaller grain sizes, which may possess higher grain boundary densities, are mathematically accounted for, ensuring that the model does not underestimate the localized corrosion kinetics.

To determine the exact tₒₓ, the computational model executes an inverse design algorithm. This algorithm iteratively adjusts the oxide layer thickness parameter within the virtual simulation, repeatedly calculating the resulting T_{d}, until the predicted degradation timeline perfectly aligns with the required bone remodeling time target (T_{b}). Furthermore, the model can generate a spatial degradation profile and a localized stress-strain distribution map, allowing engineers to visualize how the implant's mechanical integrity will evolve in vivo as the cross-sectional area diminishes.

### IV. High-Temperature Oxidation (HTO) and Controlled Cooling Protocols

Upon determining the precise tₒₓ, the physical surface modification of the magnesium alloy implant is conducted. The implant is placed into the heat treatment apparatus and subjected to the High-Temperature Oxidation (HTO) process.

The target temperature for the HTO process is strictly maintained between 400° C. and 600° C. This temperature range is highly critical; temperatures below 400° C. fail to produce a sufficiently dense or thick oxide layer within a practical timeframe, while temperatures exceeding 600° C. risk partial melting of materials, and structural distortion of the implant. The oxidation kinetics are further regulated by conducting the HTO process in a controlled gas atmosphere having a specifically restricted oxygen partial pressure combined with an argon inert gas. The inclusion of argon prevents uncontrolled exothermic oxidation (substrate ignition) while allowing a steady, homogenous growth of the MgO layer to the exact calculated thickness (tₒₓ).

Following the high-temperature dwell period, the method introduces a highly critical cooling phase. Thermal oxide layers grown on magnesium substrates inherently possess a significantly different coefficient of thermal expansion (CTE) compared to the underlying bulk magnesium metal. Conventional rapid cooling (e.g., air quenching or water quenching) induces massive residual tensile stresses at the oxide-metal interface, inevitably resulting in severe thermal shock, spallation, and catastrophic micro-cracking of the oxide film. A micro-cracked oxide layer is entirely ineffective in vivo, as physiological chloride ions will rapidly penetrate the cracks, leading to accelerated, localized pitting corrosion. To permanently resolve this, the present invention mandates cooling the surface-modified implant from the target temperature at a precisely controlled cooling rate of strictly 1° C. to 15° C. per minute. This exceedingly slow, furnace-controlled cooling protocol effectively dissipates thermo-mechanical residual stresses, ensuring that the final protective oxide layer is structurally intact and substantially free of micro-cracks.

### V. Specific Embodiments and Clinical Applications

The predictive surface modification method yields a superior biodegradable magnesium alloy implant. In certain embodiments, the magnesium alloy substrate utilized in the system comprises a microstructure that is substantially free of rare-earth element (REE) inclusions. Eliminating REEs (such as Gadolinium, Yttrium, or Neodymium) significantly enhances the long-term systemic biocompatibility of the implant and prevents the accumulation of toxic heavy metals in the patient's liver or kidneys during the degradation process.

The methodology is uniquely advantageous when applied to highly complex, porous structures having an interconnected pore network, which are notoriously difficult to protect uniformly using traditional wet-chemical or polymeric coatings.

In one specific embodiment, the implant is a high-porosity dental mesh used for alveolar ridge augmentation. The rapid degradation of bare magnesium meshes typically results in a massive local release of hydrogen (H₂) gas, leading to the formation of subcutaneous gas pockets that induce tissue necrosis, flap dehiscence, and implant failure. By utilizing the present computational HTO method, the optimal oxide layer thickness (tₒₓ) is tailored to restrict the initial hydrogen gas evolution rate to a physiologically acceptable level (a rate at which the surrounding vascular network can naturally diffuse the gas). Simultaneously, the tailored oxide layer allows the mesh to gradually release magnesium ions to actively stimulate osteogenesis (bone formation) while maintaining a stable structural integrity throughout the entire bone remodeling time target (T_{b}).

In another specific embodiment, the implant is a porous fusion prosthesis, such as a spinal interbody fusion cage or a talus replacement. In this application, the oxide layer dictates a degradation profile specifically configured to provide a gradual, continuous transfer of physiological load from the degrading metallic struts to the newly ingrown trabecular bone. This dynamic load transfer mechanism actively prevents stress shielding—a common cause of bone resorption seen with permanent titanium implants—thereby promoting a denser and healthier bone fusion mass.

### VI. Working Examples

To empirically validate the predictive capabilities of the present invention, an in vitro demonstration was conducted using a simplified solid geometry (disc/cylinder model). The required physiological target dictated a specific longevity, establishing the required T_{b}. The computational model, utilizing the physiological diffusion coefficients and the initial solid concentrations, iteratively determined that an optimal high-temperature oxidation (HTO) duration of exactly 8 hours at the target temperature would produce the required oxide layer thickness to meet the target.

Simulation results compared untreated bare magnesium specimens (0 μm oxide) against computationally modeled specimens with 5 μm and 10 μm oxide layers. The numerical model predicted a highly controlled, significant reduction in mass loss over a simulated 7-day period for the oxidized specimens. Subsequently, physical magnesium specimens were subjected to the HTO process under the calculated parameters (including the 1-15° C./min cooling rate) to grow the defined oxide layers. Empirical mass loss testing of these physical specimens in a physiological fluid environment over a 7-day period strongly correlated with the computational predictions. The physical specimen featuring the optimized oxide layer demonstrated a high degree of correlation between the in silico simulated corrosion rate and the empirical testing results, definitively validating the accuracy and reliability of integrating numerical modeling with precise thermal process control to achieve tailored implant biodegradability.

### Best Mode For Carrying Out the Invention

The best mode for carrying out the present invention involves manufacturing a highly porous biodegradable magnesium alloy implant, specifically a dental mesh or a fusion prosthesis. The preferred substrate is a biocompatible magnesium alloy, such as a rare-earth-free alloy or WE43.

The optimal execution of the method begins by defining the patient-specific bone remodeling time target (T_{b}). Subsequently, the optimal oxide layer thickness (tₒₓ) is calculated utilizing the provided computational numerical model based on the convection-diffusion equation, integrating the implant's grain size, a simulated body fluid temperature of 37° C., and a pH of 7.4.

To physically realize this optimal thickness, the implant is subjected to a High-Temperature Oxidation (HTO) process in a controlled oxygen-argon gas mixture. The most preferred thermal operating parameters include heating the implant to a target temperature between 400° C. and 600° C. for an optimal duration of approximately 8 hours. Critically, to fulfill the best mode of achieving a defect-free protective film, the HTO process must be concluded with a strictly controlled furnace cooling phase at a rate of 1° C. to 15° C. per minute. This specific cooling rate optimally dissipates thermo-mechanical residual stresses, completely preventing micro-cracking within the newly formed oxide layer. Consequently, the implant achieves the precise degradation profile required to prevent premature structural failure and limits hydrogen gas evolution during the critical bone remodeling period.

What is claimed is:

1. A method of manufacturing a biodegradable magnesium alloy implant with a controlled degradation rate, comprising: defining a bone remodeling time target (T_{b}) based on an anatomical location and a physiological requirement biological environment and load-bearing of the implant; and modifying a surface of the magnesium alloy implant by subjecting the implant to a high-temperature oxidation (HTO) process at a target temperature between 400° C. and 600° C. to form an oxide layer; characterized in that the method further comprises: (a) calculating an optimal oxide layer thickness (tₒₓ) required to achieve a predicted implant degradation time (T_{d}) that is equal to or greater than the time target (T_{b}) by utilizing a computational numerical model applying a convection-diffusion equation and Fick's first law to simulate a mass transfer of magnesium ions, wherein the numerical model uniquely integrates the pre-oxidation grain size of the magnesium alloy as a controlling parameter to counteract micro-galvanic corrosion; (b) executing the HTO process to generate the oxide layer having exactly the calculated optimal oxide layer thickness (tₒₓ); and (c) cooling the surface-modified implant from the target temperature at a precisely controlled cooling rate of 1° C. to 15° C. per minute to prevent micro-cracking within the formed oxide layer.

2. The method of claim 1, wherein the simulated mass transfer in the computational numerical model incorporates dynamic concentration condition tracking the degradation front, an initial solid concentration of magnesium (c_{solid}), and a saturation concentration of magnesium ions (cₛₐₜ) in a simulated body fluid at 37° C. and a pH of 7.4.

3. The method of claim 1, wherein calculating the optimal oxide layer thickness (tₒₓ) comprises an inverse design algorithm iteratively adjusting an oxide layer thickness parameter within the computational numerical model until the predicted implant degradation time (T_{d}) aligns with the bone remodeling time target (T_{b}).

4. The method of claim 1, wherein the high-temperature oxidation (HTO) process is performed in a controlled gas atmosphere having a specifically restricted oxygen partial pressure combined with an argon inert gas to regulate oxidation kinetics and prevent substrate ignition.

5. The method of claim 1, wherein the computational numerical model further generates a spatial degradation profile and a localized stress-strain distribution map of the implant.

6. The method of claim 1, wherein the magnesium alloy implant is a porous structure having an interconnected pore network, selected from the group consisting of a dental mesh and a fusion prosthesis.

7. The method of claim 6, wherein the porous structure is the dental mesh, and the calculated optimal oxide layer thickness (tₒₓ) restricts a hydrogen gas evolution rate to a physiologically acceptable level and maintains a stable structural integrity throughout the bone remodeling time target (T_{b}).

8. The method of claim 6, wherein the porous structure is the fusion prosthesis, and the oxide layer dictates a degradation profile configured to provide a gradual transfer of physiological load to newly formed bone, thereby preventing stress shielding.

9. The method of claim 1, wherein the computational numerical model continuously correlates the grain size with a micro-galvanic corrosion rate factor to dynamically adjust the required optimal oxide layer thickness (tₒₓ).

10. A surface-modified biodegradable magnesium alloy implant comprising: a magnesium alloy substrate; and a protective oxide layer formed on a surface of the substrate by a high-temperature oxidation (HTO) process; characterized in that the implant is manufactured by the method of claim 1, wherein the protective oxide layer has a specific optimal thickness (tₒₓ) predetermined by the computational numerical model based on the grain size of the substrate to synchronize a degradation profile of the implant with a patient-specific bone remodeling time target (T_{b}), and wherein the protective oxide layer is substantially free of micro-cracks due to the controlled cooling rate of 1° C. to 15° C. per minute.

11. The surface-modified biodegradable magnesium alloy implant of claim 10, wherein the implant is a high-porosity dental mesh comprising a tailored oxide layer configured to stimulate osteogenesis while suppressing rapid corrosion burst.

12. The surface-modified biodegradable magnesium alloy implant of claim 10, wherein the magnesium alloy substrate comprises a microstructure substantially free of rare-earth element (REE) inclusions.

13. A system for manufacturing a biodegradable magnesium alloy implant with a customized degradation rate, comprising: a computing device comprising a processor and a memory; and a heat treatment apparatus communicatively coupled to the computing device to perform a high-temperature oxidation (HTO) process at a temperature between 400° C. and 600° C.; characterized in that the processor of the computing device is configured to execute a numerical model applying a convection-diffusion equation to inversely calculate an optimal oxide layer thickness (tₒₓ) utilizing an initial grain size of the implant as an input parameter to achieve a predefined bone remodeling time target (T_{b}); and the heat treatment apparatus comprises a controller programmed to receive the calculated optimal oxide layer thickness (tₒₓ), regulate the HTO process duration to form said thickness, and engage a cooling mechanism to strictly cool the implant at a rate of 1° C. to 15° C. per minute.

14. The system of claim 13, wherein the computing device is further configured to convert patient-specific medical imaging data (CT/MRI) into the patient's bone anatomy with predefined bone remodeling time target (T_{b}) to achieve satisfactory bone healing.

15. The system of claim 13, wherein the controller of the heat treatment apparatus further regulates an oxygen partial pressure and an argon gas flow during the HTO process based on instructions received from the computing device.
